(19) United States
(12) Patent Application Publication
(10) Pub. No.: US 2009/0248088 A1
(43) Pub. Date: Oct. 1, 2009

(54) BONE ANCHORING DEVICE

(76) Inventor: Lutz Biedermann, VS-Villingen (DE)

Correspondence Address: CHRISTIE, PARKER & HALE, LLP, PO BOX 7068, PASADENA, CA, 91109-7068, US

(21) Appl. No.: 12/402,305

(22) Filed: Mar. 11, 2009

Related U.S. Application Data
(60) Provisional application No. 61/040,476, filed on Mar. 28, 2008.

(30) Foreign Application Priority Data
Mar. 28, 2008 (EP) 08 005 981.9

(51) Int. Cl. A61B 17/86; A61B 17/70

(52) U.S. Cl. 606/305; 606/264; 606/302

(57) ABSTRACT

A bone anchoring device includes a bone anchoring section for anchoring in a bone or a vertebrae, a receiving part for receiving a connection element, a connection element which is at least partly made of a flexible material, a fixation element for fixing the connection element in the receiving part, and wherein a sleeve is provided around the connection element in the receiving part.

### CROSS-REFERENCE TO RELATED APPLICATION(S)

The present application claims the benefit of U.S. Provisional Patent Application Ser. No. 61/040,476, filed Mar. 28, 2008, the contents of which are hereby incorporated by reference in their entirety, and claims priority from European Patent Application EP 08 005 981.9, filed Mar. 28, 2008, the contents of which are hereby incorporated by reference in their entirety.

### BACKGROUND

The invention relates to a bone anchoring device for the dynamic stabilization of bones or vertebrae.

EP 1 759 646 A1 discloses a spinal implant for the dynamic stabilization of vertebrae using a flexible rod which is clamped in the receiving part of a pedicle screw by means of a filling piece and a clamping element. The pedicle screw is a so-called monoaxial pedicle screw.

EP 1 795 134 A1 discloses a polyaxial screw for use with a flexible rod. A pressure element and a filling piece clamp the flexible rod between each other so that a flow of the material of the flexible rod in direction along the longitudinal axis of the rod is minimized.

The known bone anchoring devices are specifically adapted to be used with a flexible rod.

### SUMMARY

The disclosed bone anchoring device allows the use of a flexible rod together with known monoaxial or polyaxial screws which are not specifically adapted to clamp a flexible rod. The clamping of the flexible rod is achieved with known fixation elements, such as, for example, set screws.

Known monoaxial or polyaxial screws which are used with metallic rods can be upgraded so that they can be used with flexible rods.

The sleeve which clamps the flexible rod can have different dimensions in order to allow the use of rods of different diameter with one and the same receiving part.

Further features and advantages of the disclosure will become apparent from the detailed description of embodiments in conjunction with the accompanying drawings.

### BRIEF DESCRIPTION OF THE DRAWINGS

FIG. 1 shows a perspective elevational view of a bone anchoring device according to a first embodiment.

FIG. 2 shows the bone anchoring device of FIG. 1 in an assembled state.

FIG. 3 shows a sectional view of the bone anchoring device of FIG. 2 in an assembled state, the section being taken perpendicular to the longitudinal axis of the rod.

FIG. 4 shows a perspective view of a sleeve.

FIG. 5 shows a sectional view of the sleeve, the section being taken along line A-A.

FIG. 6 shows a perspective elevational view of the bone anchoring device according to a second embodiment.

FIG. 7 shows a perspective view of the bone anchoring device of FIG. 6 in an assembled state.

FIG. 8 shows a sectional view of the bone anchoring device of FIG. 7, the section being taken in a plane perpendicular to the rod axis.

### DETAILED DESCRIPTION

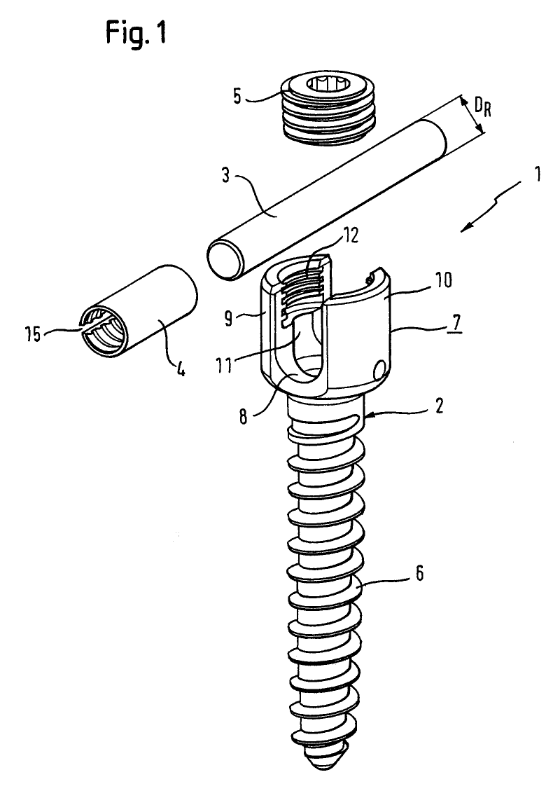

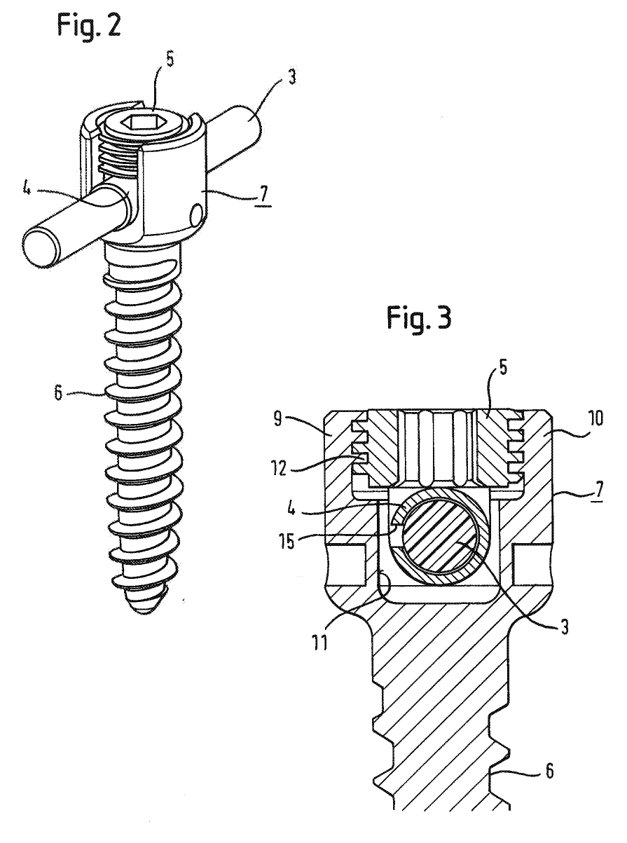

With reference to the drawings, FIGS. 1 to 3 show a first embodiment of the bone anchoring device 1 which includes a monoaxial pedicle screw with a rod. The bone anchoring device 1 includes a bone anchoring element 2, a flexible rod 3, a sleeve 4 and a fixation element 5. The bone anchoring element 2 includes a threaded shaft 6 for anchoring in the bone or in a vertebra and a receiving part 7, which is essentially cylindrical in shape and which has a coaxial bore 11 and an essentially U-shaped recess 8 forming two free legs 9 and 10. An inner thread 12 is provided at the free legs 9, 10. In the first embodiment the receiving part 7 and the threaded shaft 8 are made of one piece. The angular position of the threaded shaft 8 relative to the receiving part 7 is fixed. In the example shown in FIGS. 1-3, the threaded shaft 8 is coaxial with the receiving part 7, so that a monoaxial screw is provided. The fixation element 5 is a set screw which can be screwed in between the legs 9, 10.

The rod 3 has a generally circular cross-section and is partially or fully flexible. For example, the rod can be made of a bio-compatible plastic material, for example of an elastomer material. The material can be a polymer on the basis of polyurethane or polycarbonateurethane (PCU).

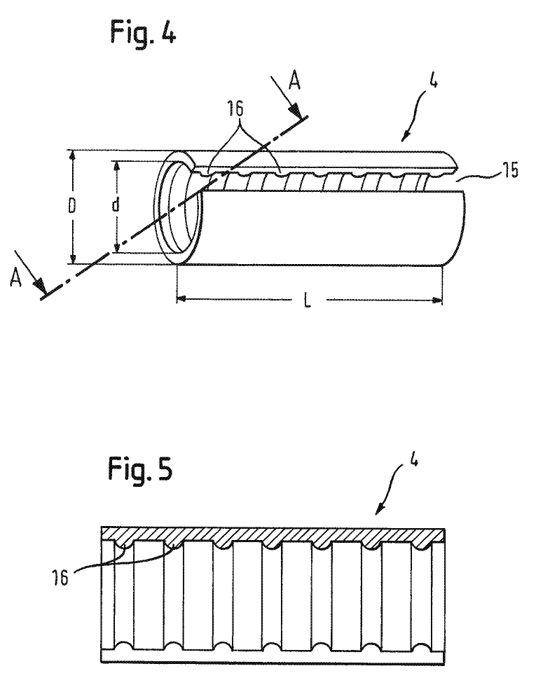

As can be seen particularly in FIGS. 1 to 4, the sleeve 4 is substantially cylindrical with an outer diameter D which is sized to fit into the U-shaped recess 8 of the receiving part 7. The inner diameter d is slightly larger than the diameter D_{R} of the rod 3 so that the sleeve can be placed onto the rod. Alternatively the size of inner diameter d of the sleeve can be equal or slightly smaller than the diameter of the rod so that the sleeve can be placed onto the rod in a pre-tensioned manner. The length L of the sleeve 4 is at least as large as the diameter of the receiving part 7 so that the sleeve rests on the bottom of the U-shaped recess when it is inserted into the receiving part 7.

The sleeve 4 further comprises a slit 15 extending from the first end to the second end. The width of the slit 15 is sized such that when the sleeve 4 surrounds the rod 3 without any pressure force acting onto the sleeve, the slit is open and has a first width. When pressure is exerted onto the sleeve 4 via the fixation element, the sleeve is compressed and the slit has a second width smaller than the first width. The sleeve 4 is made of material which is non-flexible compared to the material of the rod. For example, the sleeve 4 is made of bio-compatible metal like stainless steel or titanium or of a stiff polymer like carbon filled PEEK or other synthetic material. The slit 15 confers elasticity to the sleeve 4 in such a manner that the sleeve 4 can be compressed thereby reducing the width of the slit. The slit 15 also allows the sleeve 4 to be snapped onto the rod.

On the inner wall of the sleeve 4 a plurality of circumferentially extending rib-like protrusions 16 are provided. As shown in the embodiment, the rib-like protrusions are rounded in order not to penetrate the surface of the rod.

As can be seen in particular in FIG. 3, in the assembled state, the sleeve 4 is oriented on the rod in the receiving part 7 in such a way that the slit 15 faces towards one of the legs 9,10 of the receiving part, i.e. is substantially perpendicular to the screw axis.

In use, at least two bone anchoring elements 2 are screwed into adjacent vertebrae or bone parts. Then, the flexible rod 3 is provided with sleeves 4 at a distance corresponding to the distance of the bone anchoring elements 2. The rod 3 and a plurality of sleeves can either be preassembled or can be placed onto the rod during surgery. The sleeves 4 are displaceable along the rod axis as long as no pressure is exerted onto the sleeves. Hence, the surgeon can adjust the position of the sleeves on the rod.

Then, the rod together with the sleeves is inserted into the receiving parts of the respective bone anchoring elements and the fixation screw 5 is screwed in between the legs. When the fixation screw is tightened, it is pressed onto the sleeve as shown in FIG. 3, thereby compressing the sleeve 4 so that the width of slit 15 decreases. The materials of the rod 3 begins to flow, allowing the rib-like protrusions 16 to press onto the rod, without penetrating the internal structure of the rod, which provides a form-fit connection between the rod 3 and the inner wall of the sleeve 4. Simultaneously, the sleeve 4 is pressed onto the bottom of the U-shaped recess so that the rod is firmly held in the receiving part 7.

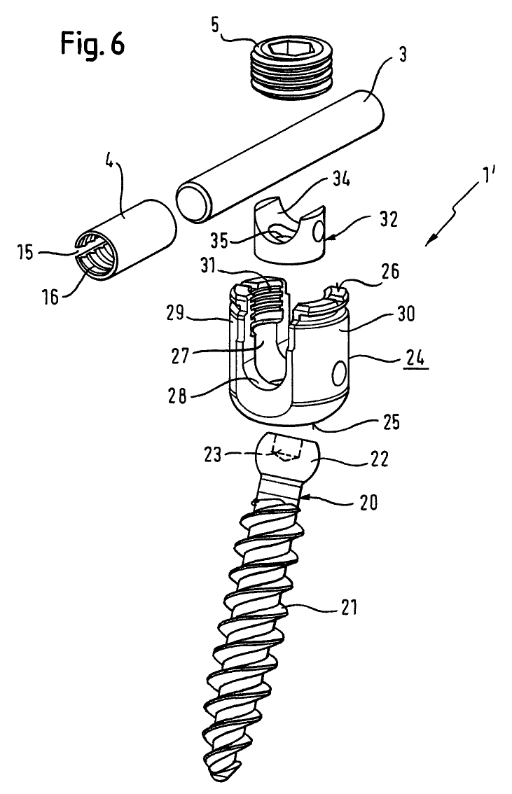

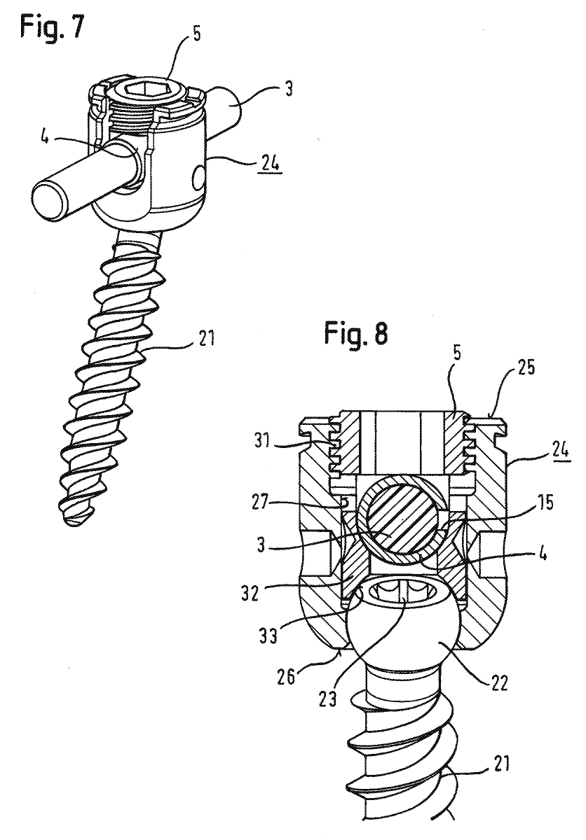

A second embodiment will now be described with reference to FIGS. 6 to 8. The second embodiment of the bone anchoring device 1′ comprises a polyaxial bone screw comprising a bone anchoring element 20 with a threaded shaft 21 and a head 22 which has the shape of a segment of a sphere. The head 22 has a recess 23 on its side facing away from the threaded shaft 21 in order to allow screwing-in of bone anchoring element.

The bone anchoring device 1′ further comprises a receiving part 24 having a first end 25 and a second end 26 opposite to the first end and a coaxial bore 27 extending from the second end 26 to the first end 25 and as shown in FIG. 8 tapering towards the second end 25 to provide a seat for the head 22. The receiving part 24 further has a substantially U-shaped recess 28 which provides a channel for receiving the rod 3. By means of the U-shaped recess 28 two free legs 29, 30 are formed which comprise an inner thread 31 cooperating with the fixation element 5.

A pressure element 32 is provided which has a generally cylindrical construction with an outer diameter, which is only slightly smaller than the inner diameter of the bore 27 to allow the pressure element 32 to be introduced into the receiving part and to be moved therein in the axial direction. On its lower side facing towards the second end 25, the pressure element 32 comprises a spherical recess 33 the radius of which corresponds to the radius of the spherical head 22 of the bone anchoring element. On the opposite side the pressure element has a cylindrical recess 34 extending transversely to the axis of the coaxial bore 27. The lateral diameter of this recess is sized such that the rod 3 with sleeve 4 can be inserted into the recess and guided therein. The depth of the cylindrical recess 34 is sized such that in an assembled state when the rod 3 with the sleeve 4 is inserted and pressed down by the fixation screw 5, the pressure element 34 exerts a pressure onto the head 22. In addition, the pressure element 32 has a coaxial bore 35 for guiding a tool therethrough.

In use the bone anchoring device can be pre-assembled in such a manner that the bone anchoring element 20 is pivotably held in the receiving part 24 and the pressure element 32 is inserted so that the cylindrical recess 34 is coaxial with the U-shaped recess 28 of the receiving part. At least two bone anchoring elements are screwed into adjacent vertebrae or bone parts and then the rod 3 with the sleeve 4 placed thereon is inserted into the U-shaped recess. The sleeve 4 is as in the first embodiment oriented in such a way that the slit 15 is oriented laterally. When the fixation screw 5 is inserted into the receiving part and tightened, it exerts pressure onto the sleeve, thereby compressing the sleeve to clamp the rod. The remaining pressure force is transferred via the sleeve to the pressure element which in turn presses onto the head 22 of the bone anchoring element to fix the head in a previously adjusted angular relationship with respect to the receiving part 24. Like in the first embodiment the rib-like protrusions 16 of the inner wall of the sleeve lead to a partially form-fit connection between the flexible rod 3 and the sleeve 4 which firmly clamps the rod and prevents a flowing of the material of the rod in a direction along the rod axis.

Several modifications of the embodiments are conceivable. Instead of the rib-like protrusions other shapes can be provided. For example the protrusions can be a plurality of small spherical projections or can have any other shape. Also a combination of projections and depressions is possible.

The inner wall of the sleeve can have a roughened surface to enhance gripping of the flexible rod. The roughened surface can be provided in addition to the protrusions or instead of the protrusions.

The wall thickness and the inner diameter of the sleeve 4 can vary so that different rods with different diameters can be placed into a receiving part of a predetermined size.

Instead of the described polyaxial screw, which is a polyaxial screw of the top-loader type, other kinds of polyaxial screws can be used such as bottom-loader types. Instead of the single fixation screw 5 any other fixation element can be used including dual-part fixation elements, outer nuts, etc. Instead of a threaded shaft, any other types of shafts like a hook, a cannulated shaft with openings, a shaft with barb elements, etc. can be used.

Instead of a rod any other type of connection element is conceivable, for example an elastomer loop being used for flexible connecting two bone anchoring elements.

While a particular form of the disclosure has been illustrated and described, it will be apparent that various modifications can be made without departing from the spirit and scope of the disclosure. Accordingly, it is not intended that the disclosure be limited, except as by the appended claims.

What is claimed is:

1. A bone anchoring device comprising:
a bone anchoring section for anchoring in a bone or a vertebrae;
a receiving part for receiving a connection element;
a connection element having at least a portion that is threadless, the threadless portion to be located in the receiving part, the threadless portion also at least partly made of a flexible material;
a fixation element for fixing the threadless portion of the connection element in the receiving part;
a sleeve to be provided around and engage the threadless portion of the connection element in the receiving part when fixing the threadless portion of the connection element in the receiving part; and
wherein the sleeve is made of a material which is rigid compared to the material of the threadless portion of the connection element.

2. A bone anchoring device according to claim 1, wherein the sleeve comprises a slit extending through a wall of the sleeve from one end of the sleeve to the opposite end of the sleeve.

3. A bone anchoring device according to claim 1, wherein an inner wall of the sleeve comprises protrusions.

4. A bone anchoring device according to claim 3, wherein the protrusions are shaped as circumferential ribs.

5. A bone anchoring device according to claim 1, wherein an inner diameter of the sleeve is slightly larger than an outer diameter of the connection element.

6. A bone anchoring device according to claim 1, wherein the sleeve is made of a rigid material.

7. A bone anchoring device according to claim 1, wherein the connection element is a rod.

8. A bone anchoring device according to claim 1, wherein the connection element is made at least partly of an elastomer material.

9. A bone anchoring device according to claim 1, wherein the receiving part is substantially cylindrical and comprises a U-shaped recess forming two free legs.

10. A bone anchoring device according to claim 1, wherein the fixation element is a locking screw.

11. A bone anchoring device according to claim 1, wherein the bone anchoring section and the receiving part are connected monoaxially.

12. A bone anchoring device according to claim 1, wherein the bone anchoring section and the receiving part are pivotably connected.

13. A bone anchoring device according to claim 2, wherein the slit is open in a direction perpendicular to a direction of a pressure force exerted by the fixation element.

14. A bone anchoring device according to claim 1, wherein an inner wall of the sleeve comprises a surface roughness.

15. A bone anchoring device according to claim 1, wherein the sleeve is made from metal.

16. A bone anchoring device comprising:
a bone anchoring section for anchoring in a bone or a vertebrae;
a receiving part for receiving a connection element;
a connection element at least partly made of a flexible material;
a fixation element for fixing the connection element in the receiving part;
a sleeve configured to be around the connection element in the receiving part;
wherein the sleeve is made of a material which is rigid compared to the material of the connection element; and
wherein the smallest inner diameter of the sleeve is larger than the largest outer diameter of the connection element.

17. A method of anchoring a bone anchoring device to bone or vertebrae, the bone anchoring device comprising a bone anchoring section for anchoring in a bone or a vertebrae, a receiving part for receiving a connection element, a connection element at least partly made of a flexible material, a fixation element for fixing the connection element in the receiving part, a sleeve around the connection element in the receiving part, and wherein the sleeve is made of a material which is rigid compared to the material of the connection element, the method comprising:
anchoring the bone anchoring section to a bone or vertebrae;
sliding the sleeve over the connection element without threading the sleeve; and
fixing the sleeve in the receiving part to fix the connection element to the receiving part.
